(19) United States
(12) Patent Application Publication
(10) Pub. No.: US 2004/0126773 A1
(43) Pub. Date: Jul. 1, 2004

(54) Assays with coded sensor particles to sense assay conditions

(76) Inventors: Oren E. Beske, Sunnyvale, CA (US); Simon Goldbard, San Jose, CA (US)

Correspondence Address: KOLISCH HARTWELL, P.C., 520 S.W. YAMHILL STREET, SUITE 200, PORTLAND, OR, 97204, US

(21) Appl. No.: 10/445,291

(22) Filed: May 23, 2003

Related U.S. Application Data
(60) Provisional application No. 60/383,092, filed on May 23, 2002.

(51) Int. Cl. C12Q 1/68; G01N 33/543

(52) U.S. Cl. 435/6; 436/523

(57) ABSTRACT

Assay systems that sense assay conditions using coded sensor particles.

### CROSS-REFERENCES TO PRIORITY APPLICATIONS

This application is based upon and claims the benefit under 35 U.S.C. § 119(e) of U.S. Provisional Patent Application Serial No. 60/383,092, filed May 23, 2002, which is incorporated herein by reference in its entirety for all purposes.

### CROSS-REFERENCES TO RELATED APPLICATIONS

This application incorporates by reference in their entirety for all purposes the following U.S. patent applications: Ser. No. 09/549,970, filed Apr. 14, 2000; Ser. No. 09/694,077, filed Oct. 19, 2000; Ser. No. 10/119,814, filed Apr. 9, 2002; Ser. No. 10/120,900, filed Apr. 10, 2002; Ser. No. 10/186,219, filed Jun. 27, 2002; Ser. No. 10/238,914, filed Sep. 9, 2002; Ser. No. 10/273,605, filed Oct. 18, 2002; Ser. No. 10/282,904, filed Oct. 28, 2002; Ser. No. 10/282,940, filed Oct. 28, 2002; Ser. No. 10/382,796, filed Mar. 5, 2003; Ser. No. 10/382,797, filed Mar. 5, 2003; Ser. No. 10/382,818, filed Mar. 5, 2003; Serial No. 10/407,630, filed Apr. 4, 2003; and Serial No. ______, filed May 23, 2003, titled MULTIPLEXED ANALYSIS OF CELLS, and naming Ilya Ravkin, Simon Goldbard, Katherine M. Tynan, Michael A. Zarowitz, and Oren E. Beske as inventors.

This application incorporates by reference in their entirety for all purposes the following U.S. provisional patent applications: Serial No. 60/426,633, filed Nov. 14, 2002; Serial No. 60/469,508, filed May 8, 2003; and Serial No. ______, filed May 22, 2003, titled MULTIPLEXED ANALYSIS OF CELLS, naming Ilya Ravkin, Simon Goldbard, Katherine M. Tynan, Michael A. Zarowitz, and Oren E. Beske as investors.

### FIELD OF THE INVENTION

The invention relates to assay systems. More particularly, the invention relates to assay systems that sense assay conditions using coded sensor particles.

### BACKGROUND

A common concern when screening for drug candidates in high-throughput screens (H TS) is whether or not all assay reagents have been added. This concern becomes more acute when a desired result in a screen is absence of signal, for example, in a screen for inhibitors of binding or activity. In such a screen, every position or well that provides no signal, a desired “negative” result in the screen, may need to be retested to determine if the result is due to addition of an effective inhibitor or is simply a false negative result. Such a false negative result may stem from a pipetting error that reduces or prevents delivery of an essential reagent. Even with improvements in automated pipetting, pipetting errors continue to be a concern as assay volumes, and thus pipetted volumes, are pushed increasingly smaller. Therefore, it may be beneficial to provide a system that determines whether all of the reagents in an assay have been properly dispensed to an assay mixture. More generally, it may be beneficial to provide a system that senses assay conditions of assays.

### SUMMARY

The invention provides assay systems that sense assay conditions using coded sensor particles.

### BRIEF DESCRIPTION OF THE DRAWINGS

FIG. 1 is a schematic representation of a method of forming of a composition for multiplexed analysis of samples and assay conditions using an array of coded particles, in accordance with aspects of the invention.

FIG. 2 is a flowchart of a method of multiplexed analysis of cells and assay conditions using an array of coded particles, in accordance with aspects of the invention.

### DETAILED DESCRIPTION

The invention provides systems, including methods, compositions, and kits, for sensing assay conditions using coded sensor particles. The sensor particles may enable detection of physical, chemical, and/or biological assay conditions. Exemplary assay conditions that may be detected with sensor particles may include the presence/absence, amount, and/or activity of a reagent in an assay mixture, the temperature of the assay mixture, and/or growth conditions within an assay mixture, among others. Each sensor particle may include a detectable code that identifies the assay condition sensed by the particle. Accordingly, sensor particles may be mixed with other coded particles (or coded carriers) for performing multiplexed sample analysis. Reading the codes of the particles may identify each type of particle in the mixture and identify the sensed condition or assay function of the particle in the analysis.

Sensor particles may include a sensor or sensor material that detects a sensed component of an assay mixture. A sensed component may be selected for assays based on availability of suitable sensor materials with which the component interacts, detectability, and effect on sample analysis in the assay. Sensed components may include ligands, epitopes, small compounds, receptors, enzymes, substrates, and/or nucleic acids, among others. Detectability of these sensed components may be intrinsic and/or extrinsic, conferred by covalently or noncovalently coupled moieties, such as dyes. Sensed components May be reagents that participate in sample analysis or may be tracer components. Tracer components may be added to reagents and/or samples prior to placing the reagents and/or samples in assay mixtures.

After placement in a reagent or sample mixture, each sensed component may allow subsequent manipulation of a reagent and/or sample mixture to be tracked during and/or after assembly of an assay mixture. Detection of one or more sensed components in the assay mixture may verify addition of the sensed components, and may indirectly report addition of other undetected reagents and/or samples previously combined with the sensed components.

Sensor particles may be used to sense more than one assay condition within a multiplexed assay. In particular, sensor particles having distinct codes and configured to detect different assay conditions may be used together. Accordingly, multiplexed assays with sensor particles may enable high-throughput screens to be performed with increased precision and confidence, reducing the need for repeating assays.

Further aspects of the invention are described in the following sections: (I) sensor particles and assay particles, (II) sensor materials, (III) assay conditions, (IV) measurement of exposure to assay conditions, (V) assays with sensor particles, and (VI) examples.

I. Sensor Particles and Assay Particles

Assays that sense assay conditions may use two types of particles or carriers, serving two distinct functions. Sensor particles may be used to sense assay conditions, such as a physical condition of an assay, or an amount or activity of a reagent, among others. Sensor particle may be configured to make no substantial contribution to experimental analysis of samples. Experimental or assay particles may be used in assay mixtures to obtain experimental results from sample analysis, for example, to measure interaction of samples and reagents, among others. Assay particles may be connected to samples and/or reagents in assay mixtures. In some embodiments, assay particles may be connected to cells (or cell populations) or may be used to measure interaction of cells with a material connected to the particles.

Both sensor particles and assay particles may have any suitable shape, size, and/or identifiable feature, based on the assay being performed.

The shape of the particles may include generally planar, cubical, cylindrical, spherical, ovalloid, and so on. Examples of suitable particles with these shapes include beads, rods, wafers, particles, sheets, and discs, among others.

The size of the particles may be selected based on one or more assay parameters, including the volume of the assay, the specific detection method, and/or the number of particles from each particle class in an assay, among others. In some applications, the particles may be larger than the wavelength of light, but smaller than the field of view (e.g., so that one or more particles may be in the field of view). In the same and/or other applications, the particles may be larger than the sample (e.g., cell), but smaller than the sample container.

Each particle may include a detectable code. The code may be different for different classes of particles to enable each class to be distinguishable. Accordingly, sensor particles may be distinguishable from assay particles. In addition, different classes of sensor particles that sense different assay conditions may be distinguishable, and different classes of assay particles that perform different sample analyses may be distinguishable. As a result, the code may identify a sensor material, a sample, and/or a reagent connected to the particle. The code may be positional and/or nonpositional and may be disposed on a portion or all of the particle. Positional codes may be formed of plural coding regions, with each region having one of plural detectable optical properties, such as plural absorption, excitation, and/or emission wavelengths.

Further examples of suitable particles (or carriers) and codes, and uses of assay particles to analyze cell populations and other analytes, are described in more detail in the patent applications identified above under Cross-References, which are incorporated herein by reference, particularly the following U.S. patent applications: Ser. No. 09/694,077, filed Oct. 19, 2000; Ser. No. 10/120,900, filed Apr. 10, 2002; Ser. No. 10/273,605, filed Oct. 18, 2002; Ser. No. 10/382,818, filed Mar. 5, 2003; and Ser. No. 10/382,797, filed Mar. 5, 2003.

II. Sensor Materials

Sensor particles may include one or more sensors or sensor materials for sensing assay conditions. The sensor material may be any compound, molecule, polymer, complex, aggregate, mixture, and/or biological entity that detectably interacts with or responds to an assay condition. Accordingly, the sensor material may be a physical sensor, a binding sensor, a chemically reactive sensor, and/or a biological sensor.

Physical sensors may include any sensor material that responds detectably to a physical condition. Accordingly, a physical sensor may respond to heat (a temperature sensor), light, pressure, particle radiation, magnetism, etc. The response may be a detectable structural change of the sensor, a change in the catalytic activity of the sensor, a change in energy absorption/emission, and/or the like.

Binding sensors may include any sensor material that binds a component of an assay mixture. The component may be a reagent used in sample analysis or a tracer used to monitor addition of a reagent mixture that includes the tracer. Binding sensors may interact with the reagent or tracer. Interaction may include any detectable effect on the binding sensor or the reagent/tracer, including specific stable and/or transient association between the binding sensor and the reagent/tracer. Stable association may be measured as complex formation of the reagent/tracer with a binding sensor that is connected to a sensor particle. Transient association may be detectable as a modification of the binding sensor, for example, when the reagent/tracer is an enzyme and the binding sensor is a substrate, or when the binding sensor is a cell(s) and the reagent/tracer binds to effect a measurable, phenotypic change in the cell(s).

A binding sensor that associates with a reagent/tracer may be a member of a specific binding pair (SBP). The SBP generally comprises any first and second SBP members that bind selectively to each other, typically with high affinity and to the exclusion of significant binding to other components of the assay mixture. Such selective binding can be characterized by a binding coefficient. Specific binding coefficients often range from about 10⁴ M to about 10⁻¹² M or 10⁻¹⁴ M and lower, and preferred specific binding coefficients range from about 10⁻⁵ M, 10⁻⁷ M, or 10⁻⁹ M and lower. Examples of SBP members include either member of the specific binding pairs listed below in Table 1. Thus, the binding member may be an antibody, an antigen, a receptor, a ligand, biotin, avidin, a single- or double-stranded nucleic acid, an enzyme, a substrate or enzyme inhibitor, polyhistidine, a molecular imprinted polymer (MIP), and/or an imprint molecule, among others.

TABLE 1
Representative Specific Binding Pairs

| First SBP Member | Second SBP Member |
|---|---|
| antigen | antibody |
| biotin | avidin or streptavidin |
| carbohydrate | lectin or carbohydrate receptor |
| DNA | antisense DNA; protein |
| enzyme substrate or inhibitor | enzyme; protein |
| polyhistidine | NTA (nitrilotriacetic acid) |
| IgG | protein A or protein G |
| RNA | antisense or other RNA; protein |
| molecular imprinted polymer (MIP) | imprint molecule |

Chemically reactive sensors may include any compound that reacts chemically with exposure to an assay condition. The compound may react with a reagent or tracer in an assay mixture, or may react with exposure to a physical condition, such as heat, light, etc. Exemplary chemically reactive pairs that may be used as chemically reactive sensors and corresponding reagents/tracers are described in U.S. patent application Ser. No. 10/407,630, filed Apr. 4, 2003, which is incorporated herein by reference.

Biological sensors may include any cell or cells that interact with, or respond to, an assay condition. For example, the cells may respond to growth conditions, the presence of a hormone or other modulator, and/or the like. The response may be a phenotypic change in the cells.

A sensor material may be included in a sensor particle by connection to the particle using any sufficiently stable association mechanism that limits separation of the sensor and particle during an assay. A suitable association mechanism may be selected based on the chemical and physical properties of each sensor material and particle. The association mechanism may be determined by a covalent bond(s) and/or noncovalent association forces, such as electrostatic attraction, hydrogen bonding, hydrophobic interactions, hydrophilic interactions, etc., between the sensor material and the particle.

The sensor material may be associated with any suitable portion of a particle. In some embodiments, the sensor material may be attached to one or more surface regions of the particle to “coat” the particle. In other embodiments, the sensor material may be incorporated into the particle during its formation, for example, when a particle is formed by stamping or molding the particle. More than one sensor material may be disposed on or in any suitable surface region or interior portion of a particle. For example, the sensor material may be disposed uniformly on surfaces of the particle, distributed throughout the particle, and/or localized to discrete portions of the particle. When the particle includes more than one sensor material, the sensor materials may be intermixed or spatially discrete. In some embodiments, the particle may include a positional array of two or more sensor materials, which are distinguishable from one another based on relative and/or absolute position within the particle. When the sensor material is a molecular imprinted polymer (MIP), the MIP may be formed during molding of the particle and may represent a substantial portion of the particle. Alternatively, the MIP may be included in a surface layer formed in situ on the particle or formed separately and applied as a film.

Further examples of sensor materials, binding members, and connection of binding members and chemically reactive members to particles are described in the patent applications listed above in the Cross-References, which are incorporated herein by reference, particularly the following U.S. patent applications: Serial No. 10/120,900, filed Apr. 10, 2002; Ser. No. 10/273,605, filed Oct. 18, 2002, and Ser. No. 10/407,630, filed Apr. 4, 2003.

III. Assay Conditions

Sensor particles may sense exposure to assay conditions. An assay condition may include any physical, chemical, and/or biological condition under which an assay is conducted to produce one or more assay results. Physical conditions may include exposure of an assay mixture to heat (temperature), light, pressure, a magnetic field, and/or an electric field, among others. Chemical conditions may include any aspect of the composition of an assay mixture, including pH, ionic strength, solvent composition, and/or the presence/amount/activity of a sensed component (see below). Biological conditions may include any aspect of the biological materials that are included in an assay mixture. The assay condition may be present for any suitable period of time during the assay.

Sensing an assay condition is not a primary purpose of the assay, but is an ancillary purpose, for example, to verify a condition of an assay or to define an assay condition to enable interpretation of assay results. Verifying a condition may result from measuring an assay condition to demonstrate that the assay condition lies within a predetermined acceptable range of assay conditions or is above a predetermined acceptability threshold. Defining an assay condition to enable interpretation of results may involve, for example, adjusting an assay result based on the defined assay condition.

Sensor particles may interact with a sensed component to verify, among others, the presence, amount, and/or activity of the sensed component. A sensed component generally comprises any molecule, complex, polymer, material, particle, and/or biological entity, among others, that interacts with a sensor particle and that is included in a reagent or sample prior to addition of the reagent or sample to an assay mixture. The sensed component may directly or indirectly report the presence/amount/activity of a reagent or a reagent mixture, or the presence of a sample, among others. Exemplary sensed components may include reagents or tracer components of reagent mixtures, among others. A reagent (or reagent mixture), as used herein, may include any compound or composition that contributes to obtaining an assay result. The reagent may interact with a sample, may facilitate or catalyze interaction, and/or the like. Exemplary reagents may include, but are not limited to, dyes, enzymes, enzyme substrates, ligands, buffers, salts, and fluids.

Suitable sensed components may be selected based on the availability of a corresponding sensor material, detectability, and/or non-interference with the assay being performed, among others. A suitable sensed component may interact with a sensor material connected to a sensor particle. Therefore, sensed components may include any member of a specific binding pair, for example, an antibody, an antigen, a receptor, a ligand, biotin, avidin, a single- or double-stranded nucleic acid, an enzyme, a substrate or enzyme inhibitor, polyhistidine, a molecular imprinted polymer, and/or an imprint molecule, among others. Alternatively, a sensed component may be a chemically reactive member of a chemically reactive pair.

The sensed component may be modified to facilitate detection when bound to, or reacted with, a sensor material of a particle. Modification may include covalent or noncovalent attachment of a label, such as a dye, a binding member that does not interact with sensor materials in an assay mixture, or an enzyme. Dyes may include luminophores/fluorophores (such as fluorescein, rhodamine, Texas red, Alexa dyes (available from Molecular Probes), phycoerythryn, GFP, and so on), chromophores (such as diazo dyes), and/or any other material that has a distinctive optical property. Suitable specific binding members or enzymes may include any of the specific binding members described above, for example, an enzyme (such as beta-galactosidase, alkaline phosphatase, chloramphenicol acetyltransfetase, luciferase, a peroxidase, and/or so on), an epitope (such as dinitrophenyl, HA-, AU1-, or myc-tag, among others), biotin, avidin, a nucleic acid, and so on.

Sensed components may be tracers or reagents. A tracer may be present at a low concentration and may not participate substantially in the assay itself to produce assay results. Such a tracer may be any material that interacts detectably with a sensor particle. Alternatively, the sensed component may be a reagent that participates in sample analysis, but which is added in sufficient excess so that its interaction with sensor particles does not substantially affect its ability to participate in sample analysis.

IV. Measurement of Exposure to Assay Conditions

Assay conditions may be detected using sensor particles, by measuring a signal corresponding to a sensed condition from the particles and reading the codes of the sensor particles to identify the sensed condition. Suitable or preferred detection methods depend on the nature of the sensed condition being detected and the type of signal produced by the sensed condition. For example, if sensed components are optically detectable, then binding of the sensed components to sensor particles may be detected by any suitable optical method.

Detection may be qualitative and/or quantitative. Qualitative detection is the determination of the presence or absence of a sensed component in an assay mixture (e.g., added or not added to the reaction mixture, or type among a plurality of possibilities). Quantitative detection may be the quantitative or semi-quantitative determination of the amount (e.g., absolute or relative number, mass, and/or concentration, among others) or activity of any sensed component present in an assay mixture, or a magnitude or value of a sensed physical condition. Quantitative detection may be useful in measuring variations in dispensing, for example, to identify assay mixtures that may produce anomalous results due to inaccurate addition of assay reagents or sample.

Assay conditions may be measured before, during, and/or after sample analysis of an assay mixture. A suitable time for detecting assay conditions may be based on how sample analysis is conducted. For example, if both sensor particles and assay particles are used to detect sensed components and to produce assay results, respectively, these particles may be analyzed in series and/or in parallel. In some embodiments, sensor particles may be analyzed before assay particles, for example, to first verify formation of a desired assay mixture. Accordingly, in some embodiments, assay particles may be analyzed only if the desired assay mixture has been formed. In some embodiments, assay particles may be analyzed before sensor particles, for example, restricting analysis of sensor particles to a particular assay result(s) obtained from the assay particles, such as a reduced signal or a negative result. The sensor particles may verify that the reduced signal or negative result was produced with a desired assay mixture (or sensed component).

Further aspects of analyzing coded particles, such as measuring sample characteristics or interactions, and reading codes, are described in the patent applications listed in the Cross-References, which are incorporated by reference herein, particularly the following U.S. patent applications: Ser. No. 09/694,077, filed Oct. 19, 2000; Ser. No. 10/120,900, filed Apr. 10, 2002; and Ser. No. 10/282,904, filed Oct. 28, 2002.

V. Assays with Sensor Particles

Sensor particles may be used in any assay for which an assay condition is measured. In particular, sensor particles may be used in assays combining two or more assay components to form an assay mixture. For example, sensor particles may be suitable for assays in which (1) two or more fluids/mixtures are combined, (2) one or more reagents/reagent mixtures are added in series and/or in parallel to an assay mixture, (3) sample preparation or sample addition is variable or unreliable, and so on. Suitable sensed components, particularly tracers, may be added to a reagent or sample at any time during the preparation of the reagent (or reagent mixture) or sample.

Sensor particles may be designed to sense one or more components of each reagent that is pipetted. The sensor particles may sense a reagent molecule itself and/or a “tracer” molecule that has been added to the reagent, typically in small amounts. In the latter scenario, a unique molecular tracer may be added to each reagent. Then, a signal measured from each sensor particle may indicate whether each reagent was added to the assay mixture.

### VI. EXAMPLES

The following examples describe selected aspects and embodiments of the invention, including systems and methods for using coded particles in nonpositional arrays to perform multiplexed assays of samples and assay conditions. These examples are included for illustration and are not intended to limit or define the entire scope of the invention.

### Example 1

### Composition for Analysis of Samples and Assay Conditions

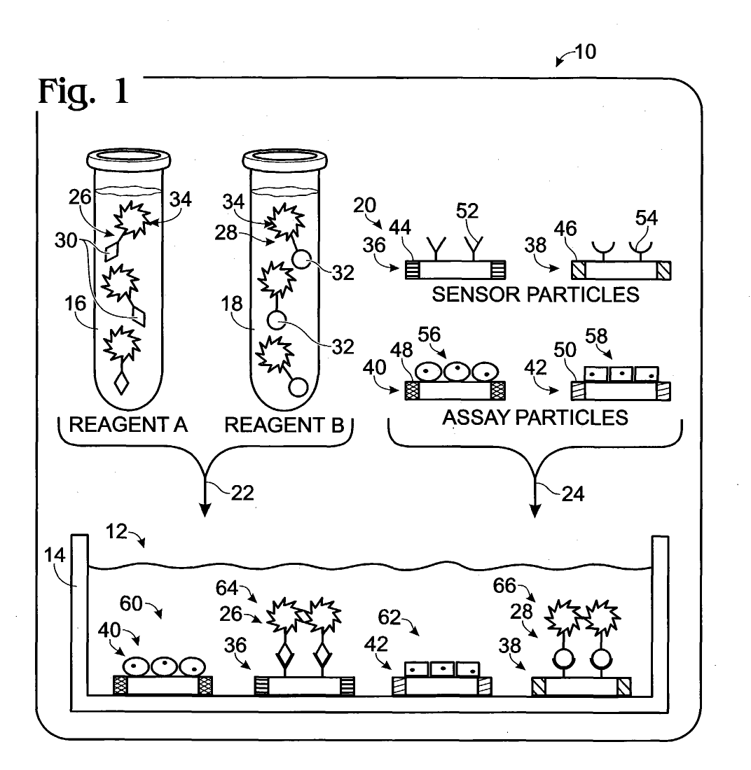

This example describes a schematic representation of a method of forming a composition for multiplexed analysis of samples and assay conditions using coded particles; see FIG. 1.

Method 10 shows formation of a composition or assay mixture 12 held by a microplate well 14. Assay mixture 12 may be formed by placing one or more reagents 16, 18 (Reagents A and B) and a set of coded particles 20 in well 14, shown at 22 and 24, respectively.

Reagents 16, 1 may include distinct tracers or tracer components 26, 28. Each tracer may include a specific binding member 30, 32 and may be detectable. The tracers may be added in minor or trace amounts to each reagent, to “spike”, the reagent, and may not be required otherwise for the assay. In the present illustration, each binding member includes a dye 34 that is detectable optically. Exemplary tracers may include dye-labeled biotin and dinitrophenyl, among others. Tracers may be distinct to enable addition of each reagent to be detected independently. However, connected dye 34 may be the same for each tracer or may be different.

Coded particles 20 may be of at least two functionally different types, sensor particles 36, 38 and assay particles 40, 42. Each type may include one or more distinguishable classes. All classes may be distinguishable using codes 44, 46, 48, and 50.

Sensor particles 36, 38 may be configured to detect one or more assay conditions, such as presence/absence, amount, and/or activity of a reagent. Accordingly, each sensor particle may include or be connected to a sensor material, such as binding partners 52, 54 of particles 36, 38, respectively. Each binding partner may be configured to bind a tracer or reagent component of reagents 16, 18. In the present illustration, binding partner 52 binds to, and thus senses, tracer 26, and binding partner 54 binds to, and thus senses tracer 28. In an exemplary embodiment, binding partner 52 may be avidin or streptavidin, and binding partner 54 may be an antibody to dinitrophenyl.

Assay particles 40, 42 may be configured to detect assay results. For example, particles 40, 42 may be connected to different cell populations 56, 58, respectively, and an assay result may involve a measured characteristic of the cell populations. Alternatively, particles 40, 42 may be connected to reagents and may interact with cells, or may be used to perform assays without cells, among others.

In assay mixture 12, the two types of particles may perform distinct functions. Samples and reagents may interact adjacent assay particles 40, 42 to provide detectable experimental results, shown at 60 and 62. In contrast, tracers 26, 28 may bind to their respective sensor particles 36, 38 to sense an assay condition, such as proper addition of reagents 16, 18, shown at 64 and 66. During signal detection, sample-reagent interaction data and tracer-binding signals may be collected from the assay particles and sensor particles, respectively. Codes read from the particles may identify the source of each signal.

In alternative embodiments, sensor particles may include molecular-imprinted polymers (MIPs) or other molecular-imprinted materials. The MIPs may be structured to bind specifically to a reagent component or tracer, among others. Accordingly, the MIPs may sense proper addition of tracer or reagent components in a multiplexed particle-based assay. Further aspects of MIPs and other molecular imprinted materials are described in U.S. patent application Ser. No. 10/273,605, filed Oct. 18, 2002, and incorporated herein by reference.

### Example 2

### Method of Multiplexed Analysis Using Sensor Particles

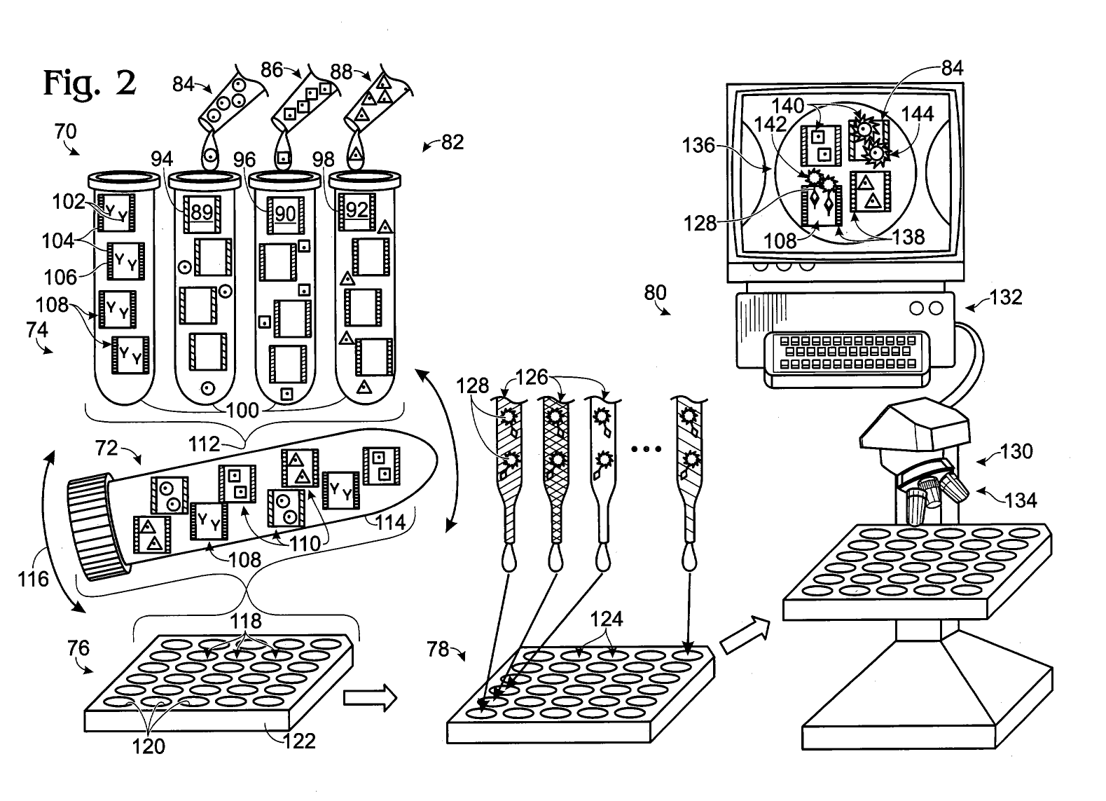

This example describes a method of multiplexed analysis of cells and one or more assay conditions using coded particles; see FIG. 2.

Method 70 may include a series of operations to achieve multiplexed analysis. A nonpositional array 72 of assay particles and sensor particles may be created, shown at 74. The nonpositional array may be placed at one or more examination sites, shown at 76. An assay mixture may be formed at each of the examination sites, shown at 78, to expose the sensor particles to one or more assay conditions. Coded particles may be analyzed, shown at 80, to provide assay results and assay conditions for the assay results.

Creating nonpositional array 72 may include connecting cells to coded particles, shown at 82. Different cell populations 84, 86, 84 may contact different classes of coded particles 89, 90, 92, respectively, to provide connection of the cells to particles. Each class of coded particle may have a different code 94, 96, 98, and may be placed in fluid isolation from other classes of particles (and other cell populations), for example, in separate vessels 100, during connection to cells.

Creating nonpositional array 72 also may include connecting a sensor material 102 to another class of coded particles 104, having a different code 106, to create sensor particles 108. In the present illustration, the sensor material is a specific binding member.

Creating nonpositional array 72 further may include mixing sensor particles 108 and assay particles 110, shown at 112. The assay particles may be produced by connection of cells to coded particles 89, 90, 92, as described above. Sensor particles and assay particles may be combined in a vessel 114, such as a screw-cap tube, and then the vessel (or fluid therein) may be agitated, vortexed, and/or inverted, among others, as shown at 116, to achieve mixing.

Placing nonpositional array 72 at examination sites 118 may be performed next. Portions of the array may be placed at each examination site 118 by dispensing aliquots of array 72. Examination sites may be any suitable vessel or surface, such as wells 120 of a microplate 122.

Forming assay mixtures 124 at the examination sites may include exposing sensor particles 108 to assay conditions. The assay conditions may be exposure to different reagents 126, such as different test compounds or drug candidates. Each reagent or reagent mixture may include a tracer 128 that binds to sensor particles 108.

Analyzing particles may include reading codes and measuring parameters from sensor particles 108 and assay particles 110. Reading and measuring may be performed using an image capture device 130 and an image analysis system 132. Image capture device 130 may include optics 134, a light source, and a detector, among others, to create at least one image 136 of particles at an examination site. The detector may include a CCD camera or array to capture code information 138 and parametrical information 140 from the particles. The image analysis system 132 may analyze image 136 to identify each class of particle based on the code information. In addition, the image analysis system may relate the parametrical information to the cell population or sensor material connected to each class of particle (and thus particle code). In the present illustration, the image analysis system may interpret a binding signal 142 from tracer 128 on sensor particle 108 as indicative of proper reagent addition. In addition, the image analysis system may interpret an interaction signal 144 from cells of cell population 84, relative to the other populations, as selective interaction with cell population 84. Further aspects of assay analysis, particularly reading and measuring, are described in U.S. Patent Application Ser. No. 10/282,904, filed Oct. 28, 2002, and incorporated herein by reference.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of performing an assay, comprising:
forming an assay mixture including coded particles of at least two classes, each class having a different code, at least one class having a sensor of an assay condition;
reading one or more codes to identify the at least one class of particle having the sensor; and
measuring exposure of the sensor to the assay condition to determine the assay condition.

2. The method of claim 1, wherein the assay condition is at least one of presence/absence, amount, and activity of a reagent.

3. The method of claim 2, wherein the step of forming the assay mixture includes a step of adding the reagent, and wherein the step of measuring verifies the step of adding.

4. The method of claim 3, wherein the step of adding the reagent includes adding a reagent mixture including a tracer component, and wherein the step of measuring verifies addition of the tracer component and thus the reagent mixture.

5. The method of claim 4, wherein the tracer component includes at least one of an optically detectable tag, an enzyme activity, and an antibody-binding site.

6. The method of claim 1, wherein exposure of the sensor to the assay condition results in at least one of binding and chemical reaction.

7. The method of claim 1, wherein the sensor is at least one of a specific binding member, a chemically reactive species, an enzyme, a molecular imprinted polymer, an enzyme substrate, and a cell population.

8. The method of claim 1, wherein the assay condition is a physical condition of the assay mixture.

9. The method of claim 1, the physical condition corresponding to at least one of heat, electricity, electromagnetic radiation, magnetism, and pressure.

10. The method of claim 1, further comprising measuring an assay result from one or more of the at least two classes of particles.

11. The method of claim 10, wherein the step of measuring an assay result is performed after the step of measuring exposure of the sensor to the assay condition, and only if the assay condition meets a predetermined criterion.

12. The method of claim 1, wherein forming the assay mixture includes at least two different classes of particles having sensors of at least two different assay conditions.

13. The method of claim 1, wherein the step of measuring provides a binding signal, the binding signal verifying addition of an assay component when above a preselected threshold signal.

14. A composition suitable for performing an assay, comprising:
a set of at least three classes of particles, each class having a different code, at least one of the classes having a sensor of an assay condition, and at least two other classes being connected to different cell populations.

15. A kit for (1) multiplexed analysis of plural samples in an assay mixture, and (2) sensing an assay condition of the assay mixture, comprising:
a set of particles, each particle of the set including a code, the set including assay and sensor particles, each of the assay particles being adapted to analyze a sample in an assay mixture, and each of the sensor particles including a first binding member, the code identifying the first binding member, wherein the code of each of the assay particles is distinct from the code of each of the sensor particles; and
at least one sensed component configured to be bound to the first binding member in an assay mixture, the at least one sensed component including at least one of an optically detectable tag, an enzyme tag, and a specific binding partner of the first binding member.
